(19) United States
(12) Patent Application Publication
(10) Pub. No.: US 2024/0254529 A1
(43) Pub. Date: Aug. 1, 2024

(54) METHOD AND SYSTEM FOR SIMULTANEOUSLY EXTRACTING POLYSACCHARIDES, POLYPHENOLS, SOD AND VC OF ROSA ROXBURGHII TRATT FROM ROSA ROXBURGHII TRATT POMACE

(71) Applicants: CHINA AGRICULTURAL UNIVERSITY, Beijing (CN); GUANGZHOU WANGLAOJI HEALTH INDUSTRY CO., LTD, Guangzhou, Guangdong (CN)

(72) Inventors: Xiaojun LIAO, Beijing (CN); Chuyuan LI, Guangzhou (CN); Huanzhi YANG, Beijing (CN); Shaoquan WENG, Guangzhou (CN); Liang ZHAO, Beijing (CN); Rongbo ZHENG, Guangzhou (CN); Yongtao WANG, Beijing (CN); Yang ZHAO, Beijing (CN)

(21) Appl. No.: 18/629,960

(22) Filed: Apr. 9, 2024

Related U.S. Application Data
(63) Continuation of application No. PCT/CN2023/072281, filed on Jan. 16, 2023.

(30) Foreign Application Priority Data
Sep. 28, 2022 (CN) 202211191416.7

(51) Int. Cl. C12P 19/04; B01D 8/00; B01D 15/42; B01D 21/26; B01D 61/02; B01D 61/14; B01D 61/58; B01D 63/10; B01D 69/02; B01D 71/02; C12N 9/02; C12P 7/02; C12P 17/04; C12P 19/14

(52) U.S. Cl. CPC C12P 19/04; B01D 8/00; B01D 15/426; B01D 21/262; B01D 61/58; B01D 63/10; B01D 69/02; B01D 71/02; C12N 9/0089; C12P 7/02; C12P 17/04; C12P 19/14; C12Y 115/01001; B01D 61/025; B01D 61/145; B01D 2325/02831; B01D 2325/02832

(57) ABSTRACT

Provided are a method and system for simultaneously extracting polysaccharides of Rosa roxburghii Tratt, polyphenols of Rosa roxburghii Tratt, superoxide dismutase (SOD) of Rosa roxburghii Tratt, and Vc from Rosa roxburghii Tratt pomace. The method includes enzymatic hydrolysis treatment, grinding treatment, ultrasonic treatment, centrifugation treatment, ceramic membrane ultrafiltration treatment, macroporous adsorption resin treatment, elution treatment, evaporation and concentration treatment, spiral-wound membrane ultrafiltration treatment, and reverse osmosis treatment.

### CROSS-REFERENCE TO RELATED APPLICATIONS

This application is a continuation of International Application No. PCT/CN2023/072281, filed on Jan. 16, 2023, which claims priority to Chinese Patent Application No. 202211191416.7, filed on Sep. 28, 2022. The disclosures of the aforementioned applications are hereby incorporated by reference in their entireties.

### FIELD

The present disclosure relates to the field of food. Specifically, the present disclosure relates to a method and system for simultaneously extracting polysaccharides, polyphenols, SOD and Vc of Rosa roxburghii Tratt from Rosa roxburghii Tratt pomace.

### BACKGROUND

Rosa roxburghii Tratt is a fruit of perennial deciduous shrub of the Rosaceae family, and it is rich in SOD, Vc, polyphenols, trace elements, and other active ingredients, and known as the “The King Fruit”. Rosa roxburghii Tratt is widely grown in southwest China, mainly in Guizhou Province, China.

At present, the development of Rosa roxburghii Tratt pomace is merely limited to dietary fiber and polysaccharide extraction, ultrafine powder of Rosa roxburghii Tratt pomace, fermented fruit vinegar, production of feed and cultivation fertilizer, etc. Rosa roxburghii Tratt pomace has great value in use, as it is rich in active ingredients such as polysaccharides, polyphenols, SOD and Vc, etc., and it also has excellent processing performance. However, most of the Rosa roxburghii Tratt pomace are discarded without being appropriately and effectively developed and utilized, resulting in serious waste of resources and environmental pollution.

Therefore, it is urgent to effectively extract the polysaccharides, polyphenols, SOD and Vc of Rosa roxburghii Tratt from the Rosa roxburghii Tratt pomace.

### SUMMARY

The present disclosure aims to solve the technical problems existing in the related art at least to a certain extent. To this end, the present disclosure provides a method and system for simultaneously extracting polysaccharides of Rosa roxburghii Tratt, polyphenols of Rosa roxburghii Tratt, SOD of Rosa roxburghii Tratt, and Vc of Rosa roxburghii Tratt from Rosa roxburghii Tratt pomace. With such method and system, the polysaccharides, polyphenols, SOD and Vc of Rosa roxburghii Tratt can be effectively extracted from Rosa roxburghii Tratt pomace with high yield, thereby efficiently utilizing the by-products of Rosa roxburghii Tratt to produce high value-added products, and thereby promoting the development of Rosa roxburghii Tratt property, which is of great significance.

The present disclosure provides a method for simultaneously extracting polysaccharides of Rosa roxburghii Tratt, polyphenols of Rosa roxburghii Tratt, SOD of Rosa roxburghii Tratt, and Vc of Rosa roxburghii Tratt from Rosa roxburghii Tratt pomace. According to an embodiment of the present disclosure, the method includes: performing enzymatic hydrolysis treatment on the Rosa roxburghii Tratt pomace to obtain an enzymatic hydrolysis product; performing grinding treatment on the enzymatic hydrolysis product to obtain a grinding slurry; performing ultrasonic treatment on the grinding slurry to obtain an ultrasonic treatment product; performing centrifugation treatment on the ultrasonic treatment product and collecting the supernatant; performing, using a ceramic membrane, ultrafiltration treatment on the supernatant to obtain a first permeate and a first retentate, the first retentate containing the polysaccharides of Rosa roxburghii Tratt; treating the first permeate with a macroporous adsorption resin to obtain a deastringent liquid of Rosa roxburghii Tratt and a used macroporous adsorption resin; performing, using ethanol solution, elution treatment on the used macroporous adsorption resin, and evaporating and concentrating the eluate, to obtain the polyphenols of Rosa roxburghii Tratt; performing, using a spiral-wound membrane, ultrafiltration treatment on the deastringent liquid of Rosa roxburghii Tratt to obtain a second permeate and a second retentate, the second retentate containing the SOD of Rosa roxburghii Tratt; and performing reverse osmosis treatment on the second permeate to collect a third retentate, the third retentate containing the Vc.

In the method according to an embodiment of the present disclosure, through the enzymatic hydrolysis treatment on the Rosa roxburghii Tratt pomace, the tissue fibers of the Rosa roxburghii Tratt can be effectively destroyed to release the active ingredients in the Rosa roxburghii Tratt pomace. The enzymatic hydrolysis product is then ground to the grinding slurry. The grinding slurry is subjected to the ultrasonic treatment, which can further break the cell walls of the Rosa roxburghii Tratt pomace to fully release the active ingredients in the Rosa roxburghii Tratt pomace. The ultrasonic treatment product is centrifuged to remove the precipitate, and the supernatant is subjected to the ceramic membrane ultrafiltration treatment. The polysaccharides of Rosa roxburghii Tratt cannot pass through the ceramic membrane due to the large molecular weight and can thus be effectively remained on the membrane, thereby collecting the retentate containing the polysaccharides. The (first) permeate is used for the subsequent processing. Based on a large number of experiments, the Applicant has found that macroporous adsorption resin has a better adsorption effect on polyphenols of Rosa roxburghii Tratt. Therefore, the macroporous adsorption resin can be used to adsorb the polyphenols of Rosa roxburghii Tratt, which can be subsequently eluted from the macroporous adsorption resin through the elution treatment. In addition, the presence of polyphenols endows the product with an astringent taste, which can be adsorbed and effectively removed by the macroporous adsorption resin. The spiral-wound membrane ultrafiltration treatment is then performed on the deastringent liquid obtained in the previous step, to effectively separate the highly active SOD. The (second) permeate is subjected to the reverse osmosis treatment to effectively separate Vc. The Applicant creatively developed the above-mentioned series of processing steps through extensive experiments, which can simultaneously extract the polysaccharides, polyphenols, SOD and Vc of Rosa roxburghii Tratt from the Rosa roxburghii Tratt pomace.

According to embodiments of the present disclosure, the above-mentioned method for simultaneously extracting the polysaccharides, polyphenols, SOD and Vc of Rosa roxburghii Tratt from the Rosa roxburghii Tratt pomace can further have the following additional technical features:

According to an embodiment of the present disclosure, the enzymatic hydrolysis treatment includes soaking the Rosa roxburghii Tratt pomace in an enzymatic hydrolysis solution; an enzyme in the enzymatic hydrolysis solution is at least one selected from cellulase and pectinase; the enzymatic hydrolysis solution has a concentration ranging from 0.1% by volume to 0.5% by volume; a solid-liquid ratio of the Rosa roxburghii Tratt pomace to the enzymatic hydrolysis solution ranges from 1:40 g/mL to 1:50 g/mL; and said soaking is performed at a temperature ranging from 30° C. to 40° C. for 10 minutes 30 minutes. The above-mentioned optimal conditions for the enzymatic hydrolysis treatment were obtained by the Applicant through extensive experiments, whereby the tissue fibers of Rosa roxburghii Tratt can be effectively destroyed and the release rate of active ingredients in Rosa roxburghii Tratt pomace can be increased.

According to an embodiment of the present disclosure, the ultrasonic treatment is performed at a power ranging from 100 W to 300 W for 30 minutes to 60 minutes; and the centrifugal treatment is performed at a rotation speed ranging from 1000 rpm to 5000 rpm for 10 minutes 30 minutes. The above-mentioned optimal conditions were obtained by the Applicant through experimental optimization, whereby the cell wall can be effectively broken to sufficiently release the active ingredients in the Rosa roxburghii Tratt pomace.

According to an embodiment of the present disclosure, the centrifugal treatment is performed at a rotation speed ranging from 1000 rpm to 5000 rpm for 10 minutes 30 minutes. In this way, the precipitate can be effectively removed and the loss of active ingredients can be reduced, which is helpful for subsequent experiments.

According to an embodiment of the present disclosure, the ceramic membrane has a pore diameter ranging from 50 kDa to 150 kDa. The above-mentioned optimal pore diameter was obtained by the Applicant through extensive experiments, whereby the polysaccharides of Rosa roxburghii Tratt can be retained and then separated and extracted. The obtained retentate containing polysaccharides of Rosa roxburghii Tratt has a high concentration of polysaccharides of Rosa roxburghii Tratt and few impurities.

According to an embodiment of the present disclosure, the macroporous adsorption resin is selected from AB-8, and an eluent is selected from food-grade absolute ethanol. As a result, polyphenols can be adsorbed, and the polyphenols adsorbed on the macroporous adsorption resin can be eluted with the absolute ethanol elution.

According to an embodiment of the present disclosure, the spiral-wound membrane has a pore diameter ranging from 10 kDa to 30 kDa. The above-mentioned optimal molecular pore diameter was obtained by the Applicant through extensive experiments, whereby highly active SOD can be retained and then separated and extracted. The obtained retentate containing SOD has a high concentration of SOD and few impurities.

According to an embodiment of the present disclosure, a reverse osmosis membrane used in the reverse osmosis treatment has a pore diameter of 0.3 to 2 nm. The above-mentioned optimal pore diameter was obtained by the Applicant through extensive experiments, with which Vc can be retained effectively.

According to an embodiment of the present disclosure, the method further includes: drying the retentate containing polysaccharides of Rosa roxburghii Tratt, the polyphenols of Rosa roxburghii Tratt, the retentate containing SOD of Rosa roxburghii Tratt, and the retentate containing Vc, respectively, to obtain polysaccharide powders of Rosa roxburghii Tratt, polyphenol powders of Rosa roxburghii Tratt, SOD powders of Rosa roxburghii Tratt, and Vc powders of Rosa roxburghii Tratt, respectively. The drying process is performed through freeze drying in a cold trap at a temperature ranging from −65° C. to −80° C. for 24 hours to 48 hours. In this way, the material liquid can be dried and the loss of nutrients can be reduced.

In another aspect of the present disclosure, the present disclosure proposes a system for implementing the method for simultaneously extracting polysaccharides of Rosa roxburghii Tratt, polyphenols of Rosa roxburghii Tratt, SOD of Rosa roxburghii Tratt, and Vc from Rosa roxburghii Tratt pomace. According to an embodiment of the present disclosure, the system includes: an enzymatic hydrolysis device configured to perform enzymatic hydrolysis treatment on the Rosa roxburghii Tratt pomace to obtain an enzymatic hydrolysis product; a grinding device connected to the enzymatic hydrolysis device and configured to perform grinding treatment on the enzymatic hydrolysis product to obtain a grinding slurry; an ultrasonic device connected to the grinding device and configured to centrifuge the ultrasonic treatment product and collect the supernatant; a ceramic membrane ultrafiltration device connected to the ultrasonic device and configured to perform, using a ceramic membrane, ultrafiltration treatment on the supernatant to obtain a first permeate and a first retentate, the first retentate containing the polysaccharides of Rosa roxburghii Tratt; a macroporous adsorption resin device connected to the ceramic membrane ultrafiltration device and configured to treat the first permeate with a macroporous adsorption resin to obtain a deastringent liquid of Rosa roxburghii Tratt and a used macroporous adsorption resin; an elution device connected to the macroporous adsorption resin device and configured to perform, using ethanol solution, elution treatment on the used macroporous adsorption resin; an evaporation and concentration device connected to the elution device and configured to evaporate and concentrate the eluate obtained by the elution treatment to obtain the polyphenols of Rosa roxburghii Tratt; a spiral-wound membrane ultrafiltration device connected to the evaporation and concentration device and configured to perform, using a spiral-wound membrane, ultrafiltration treatment on the deastringent liquid of Rosa roxburghii Tratt to obtain a second permeate and a second retentate, the second retentate containing the SOD of Rosa roxburghii Tratt; and a reverse osmosis device connected to the spiral-wound membrane ultrafiltration device and configured to perform reverse osmosis treatment on the second permeate to collect a third retentate, the third retentate containing the Vc.

The system of the present disclosure can be used for effectively extracting polysaccharides, polyphenols, SOD and Vc of Rosa roxburghii Tratt from Rosa roxburghii Tratt pomace with high yield, thereby efficiently utilizing the by-products of Rosa roxburghii Tratt to produce high value-added products, and thereby promoting the development of Rosa roxburghii Tratt property, which is of great significance.

According to an embodiment of the present disclosure, the system further includes: a drying device connected to the ceramic membrane ultrafiltration device, the evaporation and concentration device, the spiral-wound membrane ultrafiltration device, and the reverse osmosis device, respectively, the drying device being configured to dry the retentate containing polysaccharides of Rosa roxburghii Tratt, the polyphenols of Rosa roxburghii Tratt, the retentate containing SOD of Rosa roxburghii Tratt, and the retentate containing Vc, respectively, to obtain polysaccharide powders of Rosa roxburghii Tratt, polyphenol powders of Rosa roxburghii Tratt, SOD powders of Rosa roxburghii Tratt, and Vc powders of Rosa roxburghii Tratt, respectively.

It should be noted that the features and advantages described above for the method of simultaneously extracting polysaccharides of Rosa roxburghii Tratt, polyphenols of Rosa roxburghii Tratt, SOD of Rosa roxburghii Tratt, and Vc from Rosa roxburghii Tratt pomace are also applicable to this system and will not be described again here.

The present disclosure provides the following beneficial effects.

1) In the present disclosure, the technologies such as enzymatic hydrolysis (cellulase and/or pectinase), grinding, and ultrasonic crushing are jointly adopted to treat Rosa roxburghii Tratt pomace, such that the tissue fibers and cell walls of Rosa roxburghii Tratt pomace can be effectively destroyed and the active substances in Rosa roxburghii Tratt pomace can be sufficiently extracted.

2) In the present disclosure, the grinding slurry subjected to the ultrasonic extraction is centrifuged to obtain the supernatant containing the active substances of Rosa roxburghii Tratt, and the precipitate obtained by the centrifugation can be used as animal feed additive for cattle, sheep, etc., which is environmentally friendly and of great utilization rate.

3) In the present disclosure, the macroporous adsorption resin AB-8 is used to treat the supernatant, to effectively adsorb polyphenols of Rosa roxburghii Tratt and obtain deastringent liquid of Rosa roxburghii Tratt.

4) In the present disclosure, the ultrafiltration and reverse osmosis techniques are used to treat the deastringent liquid of Rosa roxburghii Tratt, to effectively separate and concentrate active ingredients of Rosa roxburghii Tratt, thereby increasing the content of nutrients, especially SOD, Vc, etc.

5) In the present disclosure, the freeze-drying process is adopted to obtain large quantities of Rosa roxburghii Tratt powders with different components and retain the original nutrients in Rosa roxburghii Tratt to the maximum extent. In addition, the powders are easy to transport and store, having the advantages of low cost, high efficiency and strong practicality, and powders are suitable for industrial production.

6) The polysaccharide powders, polyphenol powders, SOD powders and Vc powders Rosa roxburghii Tratt obtained by the method of the present disclosure have high purity and high activity, such that the nutritional value of the product is improved and the problem of loss and instability of nutrients such as Vc and SOD during processing can also be solved. At the same time, the preparation method is simple and low-cost, suitable for large-scale production. Therefore, the products prepared by the method of the present disclosure are more beneficial to human health.

Additional aspects and advantages of the present disclosure will be set forth in part in the description which follows, and in part will be obvious from the description, or may be learned by practice of the present disclosure.

### BRIEF DESCRIPTION OF THE DRAWINGS

The above and/or additional aspects and advantages of the present disclosure will become apparent and readily understood from the description of the embodiments taken in conjunction with the following drawings, in which:

FIG. illustrates a schematic flow chart of a method for simultaneously extracting polysaccharides, polyphenols, SOD, and Vc of Rosa roxburghii Tratt from Rosa roxburghii Tratt pomace according to an embodiment of the present disclosure.

### DETAILED DESCRIPTION

The technical solutions of the present disclosure are explained below with reference to examples. Those skilled in the art can understand that the following examples are only used to illustrate the present disclosure and should not be regarded as limitations on the scope of the present disclosure. The specific techniques or conditions not specified in the examples are those described in literature in the field or product instructions will be followed. The manufacturer of the reagents or instruments used, when not specified, are all conventional products that can be purchased commercially.

### Example 1

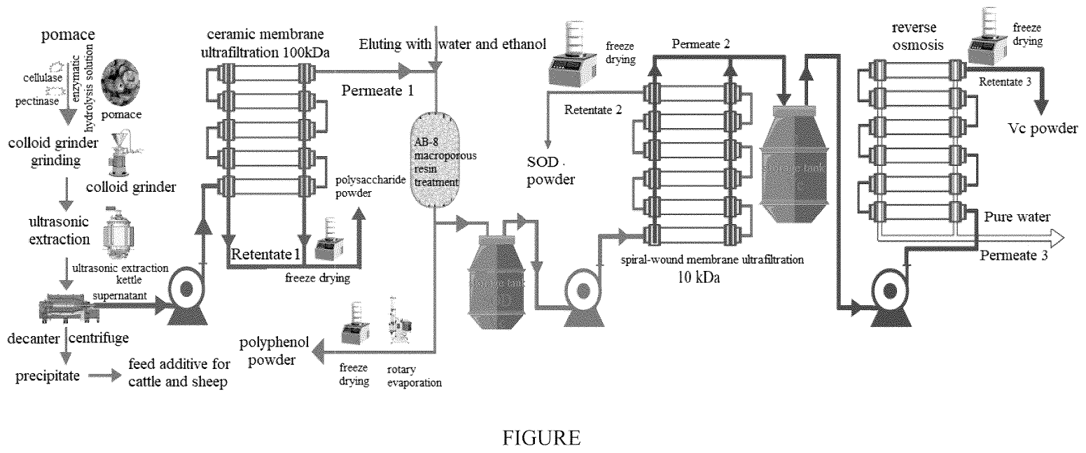

In this example, referring to FIG. 1, the following method was used to simultaneously extract polysaccharides, polyphenols, SOD and Vc of Rosa roxburghii Tratt from Rosa roxburghii Tratt pomace:
(1) Fresh, non-rotted Rosa roxburghii Tratt pomace was selected as raw material.
(2) The Rosa roxburghii Tratt pomace was soaked in an enzymatic hydrolysis solution of 0.2% by volume (a volume ratio of cellulase to pectinase of 1:1) at 40° C. for 30 minutes, in which a ratio of the Rosa roxburghii Tratt pomace to the enzymatic hydrolysis solution was 1:45 g/ml.
(3) The Rosa roxburghii Tratt pomace treated in the previous step was subjected to colloid grinding at a temperature of 40° C. to obtain grinding slurry of Rosa roxburghii Tratt, with a yield of 1 t/h.
(4) The grinding slurry was subjected to ultrasonic treatment at an ultrasonic power of 300 W for 45 min in a 100 L ultrasonic extraction tank, to obtain extracted slurry of Rosa roxburghii Tratt.
(5) The extracted slurry of Rosa roxburghii Tratt was centrifuged using a decanter centrifuge at a speed of 4000 rpm for 20 minutes to obtain a supernatant of Rosa roxburghii Tratt.
(6) The supernatant of Rosa roxburghii Tratt was subjected to ceramic membrane ultrafiltration treatment by using a ceramic membrane having a pore diameter of 100 kDa to obtain Retentate 1 (containing polysaccharides of Rosa roxburghii Tratt) and the Permeate 1.
(7) The Permeate 1 was treated with AB-8 macroporous adsorption resin in a column with a height of 2 m and an inner diameter 40 cm, to obtain a deastringent liquid of Rosa roxburghii Tratt.
(8) The polyphenols adsorbed on the resin were eluted with absolute ethanol in a volume of 2 times the column volume, and the eluate was collected and concentrated to obtain polyphenols of Rosa roxburghii Tratt, which were then concentrated using a 50 L rotary evaporator in a 50° C. water bath.
(9) The deastringent liquid of Rosa roxburghii Tratt was subjected to ultrafiltration treatment by using a spiral-wound membrane having a pore diameter of 10 kDa to obtain the Retentate 2 (containing SOD of Rosa roxburghii Tratt) and the Permeate 2.
(10) The Permeate 2 was subjected to reverse osmosis treatment by using a reverse osmosis membrane having a pore diameter of 2 nm to obtain retentate 3 (containing Vc) and permeate 3.
(11) The Retentate 1, polyphenols of Rosa roxburghii Tratt, Retentate 2, and retentate 3 were dried though freeze drying in in a cold trap at a temperature of −65° C. for 48 h, to obtain a Rosa roxburghii polysaccharide powder, a Rosa roxburghii polyphenol powder, a SOD of Rosa roxburghii Tratt powder and a Rosa roxburghii Vc powder, respectively.

### Comparative Example 1

Polysaccharide powders of Rosa roxburghii Tratt having different components were prepared according to the method of Example 1, except that in step (2), the enzymatic hydrolysis solution was replaced with drinking water, and no enzymatic hydrolysis treatment was performed.

### Comparative Example 2

The Comparative Example 2 was performed in accordance with the method of Example 1, except that the ultrasonic extraction process in step (4) was omitted.

### Comparative Example 3

The Comparative Example 3 was performed in accordance with the method of Example 1, except that the enzymatic hydrolysis treatment in step (2) and the ultrasonic extraction treatment in step (4) were not performed.

### Comparative Example 4

The Comparative Example 4 was performed in accordance with the method of Example 1, except that in step (7), the AB-8 macroporous adsorption resin was replaced with D101 macroporous adsorption resin.

The results are shown in Table 1. It can be seen that the yields of polysaccharide powders, polyphenol powders, SOD powders, and Vc powders of Rosa roxburghii Tratt prepared in Example 1 were the highest. Compared with Example 1, the yields of different powers of Rosa roxburghii Tratt obtained in Comparative Example 1 (without performing enzymatic hydrolysis), Comparative Example 2 (without performing ultrasonic treatment), and Comparative Example 3 (without performing enzymatic hydrolysis and ultrasonic treatment) decreased to varying degrees, with Comparative Example 3 decreasing the most. In Comparative Example 4 (using D101 macroporous adsorption resin), the yields of polysaccharide powders, SOD powders, and Vc powders of Rosa roxburghii Tratt were not affected, but the yield of polyphenol powders of Rosa roxburghii Tratt decreased by about 10% compared to Example 1. It indicates that the treatment using AB-8 macroporous adsorption resin in Example 1 can more effectively absorb polyphenols and increase the yield of polyphenols. Therefore, through the enzymatic hydrolysis, ultrasound, AB-8 macroporous adsorption resin treatment, ultrafiltration, reverse osmosis, freeze-drying and other processes, it is more conducive to prepare powders of different components of Rosa roxburghii Tratt from Rosa roxburghii Tratt pomace.

TABLE 1
Yields of powders of different components of Rosa roxburghii
Tratt prepared in different examples

| | Polysaccharide | Polyphenol | SOD | Vc |
|---|---|---|---|---|
| | yield | yield | yield | yield |
| Example | (g/100 g) | (mg/100 g) | (U/g) | (mg 100/g) |
| Example 1 | 3.74 | 168.58 | 187.52 | 578.34 |
| Comparative example 1 | 3.29 | 144.98 | 166.89 | 520.51 |
| (without enzymatic | | | | |
| hydrolysis) | | | | |
| Comparative example 2 | 2.99 | 134.86 | 150.02 | 462.67 |
| (without ultrasound | | | | |
| treatment) | | | | |
| Comparative example 3 | 2.06 | 92.72 | 103.14 | 318.09 |
| (without enzymatic | | | | |
| hydrolysis and ultrasound) | | | | |
| Comparative example 4 | 3.74 | 151.72 | 183.77 | 572.56 |
| (D101 macroporous | | | | |
| adsorption resin) | | | | |

In the specification, reference to the terms “one embodiment,” “some embodiments”, “an example”, “specific examples”, or “some examples” or the like means that a particular feature, structure, material or characteristic described in connection with the embodiment or example is included in at least one embodiment or example of the present disclosure. In this specification, the schematic expressions of the above terms are not necessarily directed to the same embodiment or example. Furthermore, the specific features, structures, materials or characteristics described may be combined in any suitable manner in any one or more embodiments or examples. Furthermore, those skilled in the art may combine different embodiments or examples and features of different embodiments or examples described in this specification unless they are inconsistent with each other.

Although the embodiments of the present disclosure have been shown and described above, it can be understood that the above-mentioned embodiments are illustrative and should not be construed as limitations of the present disclosure. Those of ordinary skill in the art can make changes, modifications, substitutions and variations to the above-mentioned embodiments without departing from the scope of the present disclosure.

What is claimed is:

1. A method for simultaneously extracting polysaccharides of Rosa roxburghii Tratt, polyphenols of Rosa roxburghii Tratt, superoxide dismutase (SOD) of Rosa roxburghii Tratt, and Vc of Rosa roxburghii Tratt from Rosa roxburghii Tratt pomace, the method comprising:
performing enzymatic hydrolysis treatment on the Rosa roxburghii Tratt pomace to obtain an enzymatic hydrolysis product;
performing grinding treatment on the enzymatic hydrolysis product to obtain a grinding slurry;
performing ultrasonic treatment on the grinding slurry to obtain an ultrasonic treatment product;
performing centrifugation treatment on the ultrasonic treatment product and collecting the supernatant;
performing, using a ceramic membrane, ultrafiltration treatment on the supernatant to obtain a first permeate and a first retentate, the first retentate containing the polysaccharides of Rosa roxburghii Tratt;
treating the first permeate with a macroporous adsorption resin to obtain a deastringent liquid of Rosa roxburghii Tratt and a used macroporous adsorption resin;
performing, using ethanol solution, elution treatment on the used macroporous adsorption resin, and evaporating and concentrating the eluate, to obtain the polyphenols of Rosa roxburghii Tratt;
performing, using a spiral-wound membrane, ultrafiltration treatment on the deastringent liquid of Rosa roxburghii Tratt to obtain a second permeate and a second retentate, the second retentate containing the SOD of Rosa roxburghii Tratt; and
performing reverse osmosis treatment on the second permeate to collect a third retentate, the third retentate containing the Vc.

2. The method according to claim 1, wherein the enzymatic hydrolysis treatment comprises soaking the Rosa roxburghii Tratt pomace in an enzymatic hydrolysis solution, wherein:
an enzyme in the enzymatic hydrolysis solution is at least one selected from cellulase and pectinase;
the enzymatic hydrolysis solution has a concentration ranging from 0.1% by volume to 0.5% by volume;
a solid-liquid ratio of the Rosa roxburghii Tratt pomace to the enzymatic hydrolysis solution ranges from 1:40 g/mL to 1:50 g/mL; and
said soaking is performed at a temperature ranging from 30° C. to 40° C. for 10 minutes 30 minutes.

3. The method according to claim 1, wherein:
the ultrasonic treatment is performed at a power ranging from 100 W to 300 W for 30 minutes to 60 minutes; and
the centrifugal treatment is performed at a rotation speed ranging from 1000 rpm to 5000 rpm for 10 minutes 30 minutes.

4. The method according to claim 1, wherein:
the ceramic membrane has a pore diameter ranging from 50 kDa to 150 kDa;
the macroporous adsorption resin is selected from AB-8, and an eluent is selected from food-grade absolute ethanol; and
the spiral-wound membrane has a pore diameter ranging from 10 kDa to 30 kDa; and
a reverse osmosis membrane used in the reverse osmosis treatment has a pore diameter ranging from 0.3 nm to 2 nm.

5. The method according to claim 1, further comprising:
drying the retentate containing polysaccharides of Rosa roxburghii Tratt, the polyphenols of Rosa roxburghii Tratt, the retentate containing SOD of Rosa roxburghii Tratt, and the retentate containing Vc, respectively, to obtain polysaccharide powders of Rosa roxburghii Tratt, polyphenol powders of Rosa roxburghii Tratt, SOD powders of Rosa roxburghii Tratt, and Vc powders of Rosa roxburghii Tratt, respectively,
wherein said drying is performed through freeze drying in a cold trap at a temperature ranging from −65° C. to −80° C. for 24 hours to 48 hours.

6. A system for implementing the method for simultaneously extracting polysaccharides of Rosa roxburghii Tratt, polyphenols of Rosa roxburghii Tratt, SOD of Rosa roxburghii Tratt, and Vc of Rosa roxburghii Tratt from Rosa roxburghii Tratt pomace according to claim 1, the system comprising:
an enzymatic hydrolysis device configured to perform enzymatic hydrolysis treatment on the Rosa roxburghii Tratt pomace to obtain an enzymatic hydrolysis product;
a grinding device connected to the enzymatic hydrolysis device and configured to perform grinding treatment on the enzymatic hydrolysis product to obtain a grinding slurry;
an ultrasonic device connected to the grinding device and configured to perform ultrasonic treatment on the grinding slurry to obtain an ultrasonic treatment product;
a decanter centrifugal device connected to the ultrasonic device and configured to perform centrifugation treatment on the ultrasonic treatment product and collect the supernatant;
a ceramic membrane ultrafiltration device connected to the decanter centrifugal device and configured to perform, using a ceramic membrane, ultrafiltration treatment on the supernatant to obtain a first permeate and a first retentate, the first retentate containing the polysaccharides of Rosa roxburghii Tratt;
a macroporous adsorption resin device connected to the ceramic membrane ultrafiltration device and configured to treat the first permeate with a macroporous adsorption resin to obtain a deastringent liquid of Rosa roxburghii Tratt and a used macroporous adsorption resin;
an elution device connected to the macroporous adsorption resin device and configured to perform, using ethanol solution, elution treatment on the used macroporous adsorption resin;
an evaporation and concentration device connected to the elution device and configured to evaporate and concentrate the eluate obtained by the elution treatment to obtain the polyphenols of Rosa roxburghii Tratt;
a spiral-wound membrane ultrafiltration device connected to the evaporation and concentration device and configured to perform, using a spiral-wound membrane, ultrafiltration treatment on the deastringent liquid of Rosa roxburghii Tratt to obtain a second permeate and a second retentate, the second retentate containing the SOD of Rosa roxburghii Tratt; and
a reverse osmosis device connected to the spiral-wound membrane ultrafiltration device and configured to perform reverse osmosis treatment on the second permeate to collect a third retentate, the third retentate containing the Vc.

7. The system according to claim 6, further comprising:
a drying device connected to the ceramic membrane ultrafiltration device, the evaporation and concentration device, the spiral-wound membrane ultrafiltration device, and the reverse osmosis device, respectively, the drying device being configured to dry the retentate containing polysaccharides of Rosa roxburghii Tratt, the polyphenols of Rosa roxburghii Tratt, the retentate containing SOD of Rosa roxburghii Tratt, and the retentate containing Vc, respectively, to obtain polysaccharide powders of Rosa roxburghii Tratt, polyphenol powders of Rosa roxburghii Tratt, SOD powders of Rosa roxburghii Tratt, and Vc powders of Rosa roxburghii Tratt, respectively.
